(19) United States
(12) Patent Application Publication
(10) Pub. No.: US 2025/0082583 A1
(43) Pub. Date: Mar. 13, 2025

(54) MICROCAPSULE AND PREPARATION METHOD AND USE THEREOF

(71) Applicants: INFINITUS (CHINA) COMPANY LTD., Guangdong (CN); KOREA ADVANCED INSTITUTE OF SCIENCE AND TECHNOLOGY, Daejeon (KR)

(72) Inventors: Meiling TAI, Guangdong (CN); Wanzhao LI, Guangdong (CN); Shin-Hyun KIM, Daejeon (KR); Wahyu Martumpal HAMONANGAN, Daejeon (KR)

(73) Assignees: INFINITUS (CHINA) COMPANY LTD., Guangdong (CN); KOREA ADVANCED INSTITUTE OF SCIENCE AND TECHNOLOGY, Daejeon (KR)

(21) Appl. No.: 18/505,079

(22) Filed: Nov. 8, 2023

(30) Foreign Application Priority Data
Sep. 8, 2023 (CN) 202311160919.2

(51) Int. Cl. A61K 9/50; A23L 27/00; A23L 29/00; A23L 29/30; A61K 8/60; A61K 8/81; A61K 8/891; A61K 8/893; A61K 9/48; A61Q 19/00; B01J 13/10

(52) U.S. Cl. CPC A61K 9/5031; A23L 27/72; A23L 29/035; A23L 29/30; A61K 8/60; A61K 8/8129; A61K 8/891; A61K 8/893; A61K 9/4858; A61K 9/4866; A61K 9/5089; A61Q 19/00; B01J 13/10

(57) ABSTRACT

A microcapsule and a preparation method and use thereof are provided. The microcapsule includes a core phase and a shell phase with a volume ratio of (3.4-3.8):(0.8-1.2); wherein the core phase includes 8 wt % to 12 wt % of PVA, 4 wt % to 6 wt % of a density enhancer, 0.1 wt % to 0.3 wt % of an active substance, and a residual amount of water; the shell phase includes an elastomer precursor, a curing agent and silicone oil with a mass ratio of (9-11):(0.8-1.2):(2.5-3). The microcapsules of the present invention release active ingredients completely in milliseconds, and the release process exerts a relatively strong mechanical stimulation intensity on the surrounding environment which can further improve an absorption efficiency to the active ingredients in the fields of pharmaceuticals and cosmetics, and can provide a unique sensual experience in the field of food.

### CROSS-REFERENCE TO RELATED APPLICATION

This application claims the priority benefit of China application serial no. 202311160919.2, filed on Sep. 8, 2023. The entirety of the above-mentioned patent application is hereby incorporated by reference herein and made a part of this specification.

### TECHNICAL FIELD

The present invention relates to the technical field of microcapsule preparation, and specifically relates to a microcapsule and a preparation method and use thereof.

### BACKGROUND

Microencapsulation is a technology of encapsulating a trace amount of substance inside a polymer membrane, being a micro packaging technology for storing solid, liquid and gas. Specifically, microencapsulation refers to encapsulating a target substance (core or internal phase) with a continuous membrane made of various natural or synthesized macromolecular compounds (wall or external phase) entirely, without damaging the original chemical property of the target substance, and then enabling functions of the target substance to be presented at exterior through the gradual external stimulus or slow-release effect, or protecting the core by the shielding effect of capsule wall.

At present, microencapsulation is widely used in the fields of cosmetics, pharmaceuticals and food. Generally, microcapsules are added in products of cosmetic field such as toner, lotion, serum, cream, mask, foundation, eye shadow, and face powder, for encapsulating the active ingredients by using the microencapsulation technology to prevent mutual interference between each component and to improve the stability of the active ingredients. In the field of pharmaceuticals, microencapsulation is generally used to prevent the active ingredients from being inactivated, or to conceal unpleasant smell of the active ingredients so as to enhance medication adherence of patients. In the field of food, microencapsulation is generally used to integrate the natural flavor ingredients, physiological active substances, etc. into the food system and to maintain the physiological activity.

In order to achieve rapid release of the active ingredients inside the microcapsule, self-destruct or self-rupture microcapsules arise. However, during the release of active ingredients by most of the microcapsules, release speed thereof is limited due to the slow diffusion of active ingredients instead of rapid convection, and it takes seconds or even minutes to complete the release. For example, it takes seconds for microcapsules with polyelectrolyte membrane in prior art to release the active ingredients, with mild explosion process and weak mechanical stimulation intensity to the surrounding environment, and failing to further facilitate the absorption to the active ingredients or to provide a unique sensual experience by the ultrafast release and mechanical stimulation.

### SUMMARY

In order to overcome the deficiencies in the prior art, the present invention provides a microcapsule which releases the active ingredients completely in milliseconds, and the release process exerts a relatively strong mechanical stimulation intensity on the surrounding environment which can further improve an absorption efficiency to the active ingredients in the fields of pharmaceuticals and cosmetics, and can provide a unique sensual experience in the field of food.

The first objective of the present invention is to provide a microcapsule.

The second objective of the present invention is to provide a preparation method for the above-mentioned microcapsule.

The third objective of the present invention is to provide use of the above-mentioned microcapsule in the fields of cosmetics, pharmaceuticals and/or food.

The objectives of the present invention are realized by the following technical solutions:

The present invention provides a microcapsule, which includes a core phase and a shell phase with a volume ratio of (3.4-3.8):(0.8-1.2);
wherein the core phase includes 8 wt % to 12 wt % of polyvinyl alcohol (PVA), 4 wt % to 6 wt % of a density enhancer, 0.1 wt % to 0.3 wt % of an active substance, and a residual amount of water; the shell phase includes an elastomer precursor, a curing agent and silicone oil with a mass ratio of (9-11):(0.8-1.2):(2.5-3);

The microcapsules of the present invention are capable of not only keeping a relatively high mechanical stability without any mechanical stress and storing the active substance within an interior cavity without leakage, but also completely releasing the active substance in milliseconds when the mechanical stress exceeds a critical stress of rupture of the microcapsule, and meanwhile the release process exerts a relatively strong mechanical stimulation intensity on the surrounding environment which can further improve an absorption efficiency to the active substance in the fields of pharmaceuticals and cosmetics, and can provide a unique sensual experience in the field of food. The microcapsule of the present invention is highly perceptible in terms of touch and vision, and no foreign matter sensation is felt after rupture since the microcapsule is soft and elastic, without causing any discomfort to users, further enhancing a visual aesthetic value of the end product.

Preferably, the volume ratio of the core phase to the shell phase is 3.6:1.

Preferably, the core phase includes 10 wt % of PVA, 5 wt % of the density enhancer, 0.2 wt % of the active substance, and a residual amount of water.

Preferably, shell phase includes the elastomer precursor, the curing agent and silicone oil with a mass ratio of 10:1:2.75. After curing the elastomer precursor, an elastic shell is formed which can preserve the active substance within the interior cavity of the microcapsule.

Preferably, the osmotic pressure of the core phase and the continuous phase is controlled by an osmotic pressure conditioning agent such as a sodium chloride solution. 1 to 1.1 mol/L sodium chloride solution is the most preferred.

Preferably, the core phase has an osmotic pressure of 2200 to 2280 mOsm/L, and more preferably 2240 mOSM/L.

Preferably, the density enhancer is one or more of sucrose, glucose, maltose, fructose, glycerol, or PEG. Sucrose is the most preferred.

Preferably, the elastomer precursor is polydimethylsiloxane (PDMS). PDMS is suitable for microfluidic operation for its low enough viscosity, and may provide a stable elastomer for the microcapsule after curing. Besides, PDMS has an excellent biocompatibility with silicone oil, which is suitable to be used as a raw material for cosmetics, pharmaceuticals and food.

Preferably, the curing agent is a reagent capable of curing the elastomer precursor, such as a reagent that can cure PDMS, and preferably a reagent that can cure PDMS within only 2.5 to 3.5 h under the condition of 45 to 55° C.

Preferably, the microcapsule has an inflated degree α more than 1.63, more preferably 2.53-3.59.

The term inflated degree refers to a volume ratio of an inflated microcapsule to a microcapsule before inflation. Inflated microcapsule allows its elastic shell to still maintain integrity and mechanical performance without any mechanical stress, and thus the active substance can be stored safely within the interior cavity without leakage. When deformation exceeds the critical value of mechanical rupture of the elastic shell (for example, when applying or chewing the product containing the microcapsules, mechanical stress received by the elastic shell would exceeds the critical stress of rupture of the microcapsule), the microcapsules are ruptured, release the active substance of the core phase in milliseconds intensively, and stimulate the surrounding environment mechanically, then recover to a non-stress state. Moreover, the rapid retraction process of the elastic shell will convert its own elastic potential energy into huge mechanical energy, and the ultrafast release and mechanical stimulation further improve the absorption efficiency to the active ingredients, which can further improve an absorption efficiency to the active ingredients in the fields of pharmaceuticals and cosmetics, and can provide a unique sensual experience in the field of food. The critical strain and stress that cause the rupture of microcapsule decrease with the increase of an inflated degree of the elastic shell of the microcapsule, and the higher the inflated degree of the elastic shell, the faster the release speed of the microcapsule, and the greater the mechanical stimulation to the surrounding environment.

The present invention also provides a preparation method for the above-mentioned microcapsule, wherein double-emulsion drops are prepared first by means of a microfluidic device, and then undergo a heat treatment to obtain the microcapsule.

Preferably, the microfluidic device includes an injection capillary, a collection capillary, and a square capillary at the outermost, wherein the injection capillary and the collection capillary are inserted into both sides of the square capillary, respectively, and spaced apart to form an emulsifying region in which ends of the injection capillary and the collection capillary are cone-shaped.

The microfluidic device of the present invention includes glass capillaries. The injection capillary and the collection capillary may be cylinder-shaped capillaries, and the cone-shaped end of the injection capillary is arranged coaxially opposite to the cone-shaped end of the collection capillary, so that a configuration of tip to tip is formed in the square capillary. Via such device, the microcapsules with a more uniform dimension are prepared.

Further preferably, the core phase is injected through the injection capillary, the shell phase is injected through a gap between the injection capillary and the square capillary, and the continuous phase is injected through a gap between the collection capillary and the square capillary. The continuous phase comprises 8 wt % to 12 wt % of PVA (preferably 10 wt %), and a residual amount of water.

Further preferably, a flow rate for the core phase is 170 to 180 μL/h, a flow rate for the shell phase is 170 to 180 μL/h, and a flow rate for the continuous phase is 5800 to 6200 μL/h, most preferably 175 μL/h, 175 μL/h and 6000 μL/h, respectively. The flow rates are controlled by means of an injection pump.

Preferably, the heat treatment is performed at 45° C. to 55° C. for 2.5 to 3.5 h, more preferably at 50° C. for 3 h. The heat treatment is to cure the PDMS in the raw material of the shell phase, thereby forming a semipermeable elastic shell.

Further preferably, the heat treatment is performed with stirring, for example, stirring at a rotational speed of 130 to 170 r/min. The stirring may prevent the double-emulsion drops from aggregating resulted by the hydrophobic property thereof, and may better prevent uneven shell thickness resulted by gravity.

Preferably, the microcapsules undergo an inflating treatment after the heat treatment.

Further preferably, the inflating treatment is as follows: transferring the microcapsules to an aqueous solution containing 1.5 wt % to 2.5 wt % of PVA (preferably 2 wt %), and adjusting an osmotic pressure of the aqueous solution till the microcapsules have an inflated degree α more than 1.63 (for example, by adding a 110 to 130 mmol/L sodium chloride solution). Since the osmotic pressure of the aqueous solution is much less than the osmotic pressure of the core phase of the microcapsules, and internal flow is formed in water, an osmotic pressure difference enables the microcapsules to be inflated. The inflated degree depends on a balance between the osmotic pressure difference and a tension of shell. The inflated microcapsules have a coefficient of variation (CV). It is indicated that the microcapsules are highly monodispersed for the CV of the microcapsules is low and the particle size of the microcapsules is relatively uniform.

The microcapsules of the present invention are capable of not only keeping a relatively high mechanical stability without any mechanical stress and storing the active substance within an interior cavity without leakage, which can serve as an excellent delivery carrier for active substance, but also completely releasing the active substance in milliseconds when the mechanical stress exceeds the critical stress of rupture of the microcapsule, and meanwhile the release process exerts a relatively strong mechanical stimulation intensity on the surrounding environment which can further improve an absorption efficiency to the active substance in the fields of pharmaceuticals and cosmetics, and can provide a unique sensual experience in the field of food.

Use of the above-mentioned microcapsules in the fields of cosmetics, pharmaceuticals and/or food shall be within the scope of protection of the present invention.

The present invention has the following beneficial effects:

The microcapsules of the present invention are capable of not only keeping a relatively high mechanical stability without any mechanical stress and storing the active substance within an interior cavity without leakage, but also completely releasing the active substance in milliseconds when the mechanical stress exceeds the critical stress of rupture of the microcapsule, and meanwhile the release process exerts a relatively strong mechanical stimulation intensity on the surrounding environment which can further improve an absorption efficiency to the active substance in the fields of pharmaceuticals and cosmetics, and can provide a unique sensual experience in the field of food. Therefore, the microcapsules can serve as an excellent delivery carrier for active substance, and suitable for preparing cosmetics, pharmaceuticals and food.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1a shows a schematic diagram of forming double-emulsion drops in a microfluidic device and an optical microscope image; FIG. 1b is an optical microscope image of double-emulsion drops in Step 5; FIG. 1c is a schematic diagram of preparation of microcapsules in Step 6; and FIG. 1d is a SEM image of the microcapsules obtained in Step 6.

FIG. 2a is a schematic diagram of a whole process of microcapsules from being uninflated, to being inflated, to being compressed, and to being ruptured; FIG. 2b shows an optical microscope image of the uninflated microcapsules and a photograph of an uninflated balloon; FIG. 2c shows an optical microscope image of the inflated microcapsules and a photograph of an inflated balloon;

FIG. 2d shows an optical microscope image of microcapsules being compressed by a glass slide and a photograph of a balloon being compressed; and FIG. 2e shows an optical microscope image of the ruptured microcapsules and a photograph of an exploded balloon.

FIG. 3a is a schematic diagram of a mechanical test of a microcapsule; FIG. 3b is optical images of deformation process of the microcapsule with various displacement; FIG. 3c shows force-displacement curves of the microcapsule being compressed and relaxed in various displacement; FIG. 3d is a resulting view of threshold strain and stress of rupturing the microcapsule for 12 independent microcapsules with α=2.53; and FIG. 3e show an still-shot image of the microcapsule during rupture captured by high-speed camera.

FIG. 4a shows curves of compression force-strain at various degree of inflation; FIG. 4b shows optical images of deformation process of a microcapsule having α of 1 and α of 3.59; FIG. 4c shows a histogram of an average strain to the degree of inflation; FIG. 4d shows a histogram of stress at rupture to the degree of inflation; FIG. 4e shows an affecting result of the inflated degree on a rupture time and a mechanical potential energy resulted by the inflation; FIG. 4f force-strain curve of pristine and 5-months-old microcapsules; and FIG. 4g shows average strain (left y-axis) and stress (right y-axis) at popping for the pristine and 5-months-old microcapsules.

### DETAILED DESCRIPTION OF EMBODIMENTS

The present invention is further explained in conjunction with the accompanying drawings and specific embodiments, but the present invention is not limited in any form by the embodiments. Unless specified, the reagents, methods, and equipment used in the present invention are conventional reagents, methods, and equipment in the art.

Unless specified, the reagents and materials used in the following embodiments are commercially available.

### Embodiment 1 Preparation of Microcapsules

Step 1, preparation of a core phase: preparing a first aqueous solution containing 10 wt % PVA (having a degree of hydrolysis of 87% to 89%, and a weight-average molecular weight of 13000 to 23000), 5 wt % sucrose (from Sigma-Aldrich) and 0.2 wt % pollution-free food colorant (serving as an active substance, i.e. content), and adjusting the first aqueous solution to have an osmotic pressure of 2240 mOSM/L by adding a 1 mol/L sodium chloride solution (from Junsei);

Step 2, preparation of an shell phase: mixing PDMS (Sylgard 184 from Dow Chemical), a curing agent (Sylgard 184 from Dow Chemical) and silicone oil (having a viscosity of 20 mPa's from Sigma-Aldrich) according to a mass ratio of 10:1:2.75, uniformly;

Step 3: preparation of a continuous phase: preparing a second aqueous solution containing wt % PVA;

Step 4, preparation of a microfluidic device: taking two cylinder-shaped glass capillaries (1B100F-6, from World Precision Instruments) as an injection capillary and a collection capillary, respectively, and one square glass capillary (having an inner diameter of 1.05 mm, from Atlantic International Technologies), pulling one end of the two cylinder-shaped glass capillaries into a cone-shaped end, respectively, by using a puller (P97, from Sutter Instrument), and then polishing the cone-shaped ends respectively so as to obtain required orifice diameters with abrasive paper, the injection capillary orifice diameter is 100 μm and the collection capillary orifice diameter is 200 μm; treating the injection capillary with octadecyltrimethoxysilane (from Sigma-Aldrich) to enable a surface of the injection capillary to be hydrophobic, treating the collection capillary with trimethoxy-[3-(2-methoxyethoxy) propyl]silane (from Gelest Inc.) to enable a surface of the collection capillary to be hydrophilic, and after treating for 30 min respectively, washing the injection capillary with isopropanol and the collection capillary with distilled water; upon fully drying, inserting the cone-shaped ends of the injection capillary and the collection capillary into both sides of the square capillary, respectively, and spacing apart to form an emulsifying region, so that the injection capillary and the collection capillary are arranged coaxially opposite to each other with a tip to top distance of 100 μm;

Step 5, preparation of double-emulsion drops: injecting the core phase obtained in Step 1 through the injection capillary, injecting the shell phase obtained in Step 2 through a gap between the injection capillary and the square capillary, injecting the continuous phase obtained in Step 3 through a gap between the collection capillary and the square capillary, and setting flow rates of the core phase, the shell phase and the continuous phase to 175 μL/h, 175 μL/h and 6000 μL/h, respectively, by means of an injection pump (Legato 100, from KD Scientific); the obtained double-emulsion drops at the moment have an average inner diameter of 199 μm, a coefficient of variation (CV) of 1.8% and a shell thickness of 7.8 μm; and

Step 6, preparation of microcapsules: collecting the double-emulsion drops obtained in Step 5 into a flask for water bath incubation at 50° C. for 3 h under a condition of 150 r/min so as to solidify the PDMS shell.

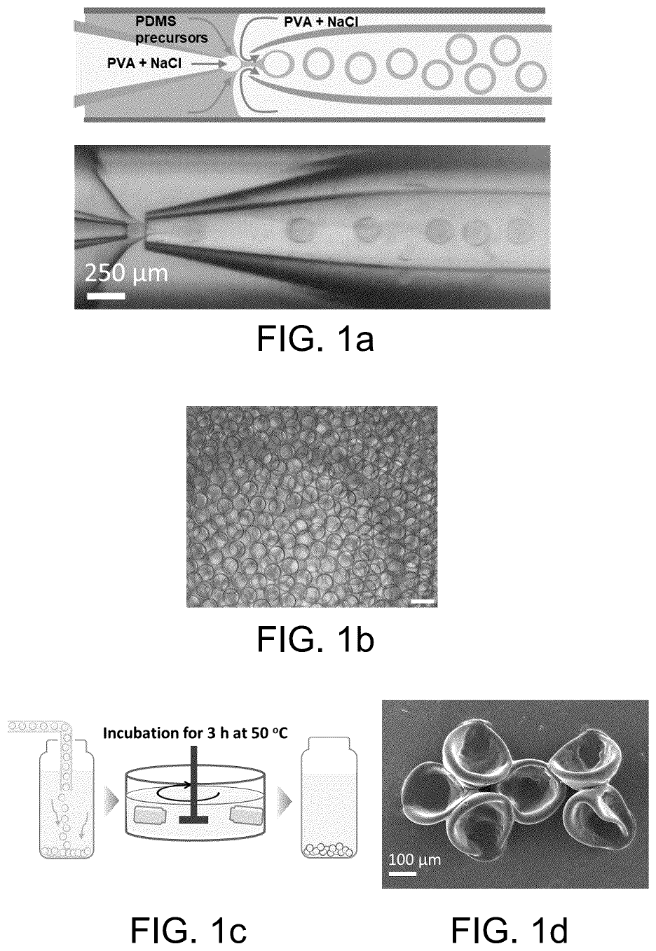

Forming process of the double-emulsion drops inside the microfluidic device was observed via an inverted optical microscope (Eclipse TS100, from Nikon) and a high-speed camera (Phantom v7.3, from Vision Research Inc.), shown as FIG. 1a. Then, the double-emulsion drops obtained in Step 5 were observed via the inverted optical microscope, an obtained optical microscope image is shown in FIG. 1b, and it can be seen that the double-emulsion drops encapsulate the content (i.e. pollution-free colorant) within the interior cavity. A schematic diagram of preparation of microcapsules in Step 6 is shown in FIG. 1c. A SEM image of the microcapsules obtained in Step 6 in shown in FIG. 1d, and it can be seen that the microcapsules have uniform dimension.

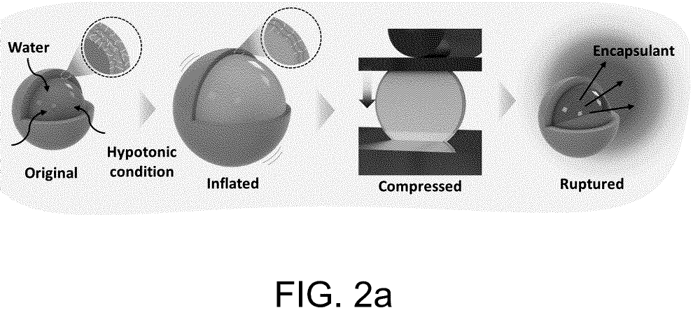

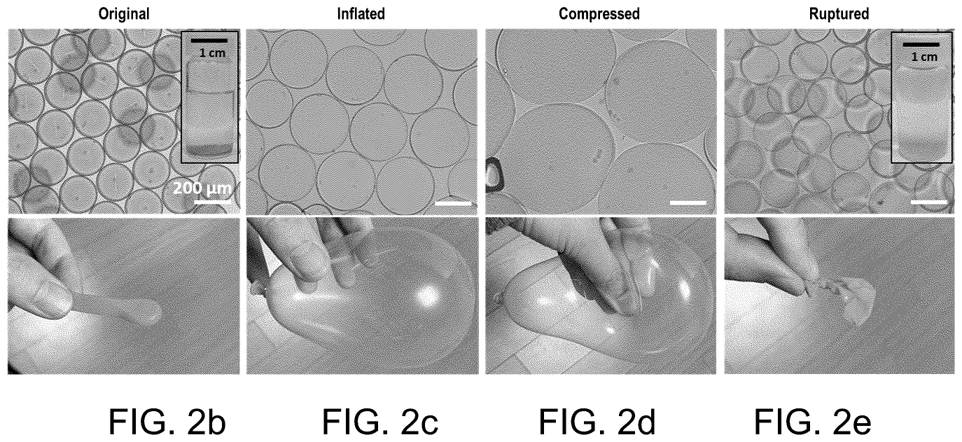

The microcapsules obtained in Step 6 were transferred to an aqueous solution containing 2 wt % PVA, and the aqueous solution was adjusted to have an osmotic pressure of 250 mOSM/L by adding a 120 mmol/L sodium chloride solution, so that the microcapsules underwent a permeable inflation in the environment having an osmotic pressure difference ΔC of 1990 mOsml⁻¹. The inflated microcapsules have an average diameter of 306 μm, a coefficient of variation (CV) of 0.8% and a shell thickness of 1 μm. The microcapsules were compressed by a pair of glass slides and induced to be ruptured so as to rapidly release the content (i.e. pollution-free colorant). Such process is shown in FIG. 2a, observed via the optical microscope, and well interpreted by the corresponding states of a balloon which are shown in FIG. 2b to FIG. 2e, presenting the microcapsule change images and the corresponding balloon images. Particularly, FIG. 2b shows an optical microscope image of the microcapsules obtained in Step 6 (uninflated) and a photograph of a balloon in normal state (uninflated); FIG. 2c shows an optical microscope image of the inflated microcapsules and a photograph of an inflated balloon; FIG. 2d shows an optical microscope image of microcapsules being compressed by a glass slide and a photograph of a balloon being compressed; and FIG. 2e shows an optical microscope image of the ruptured microcapsules and a photograph of an exploded balloon. Illustrations in FIG. 2b and FIG. 2e are photographs of a suspension liquid containing the microcapsules, indicating the uninflated state of FIG. 2b to the ruptured state of FIG. 2e, where the microcapsules released the content (i.e. pollution-free colorant) from the interior cavity.

### Embodiment 2 Preparation of Microcapsules

Step 1, preparation of a core phase: preparing a first aqueous solution containing 8 wt % PVA, 6 wt % sucrose and 0.1 wt % pollution-free food colorant, and adjusting the first aqueous solution to have an osmotic pressure of 2280 mOSM/L by adding 1.1 mol/L sodium chloride solution; Step 2, preparation of a shell phase: mixing PDMS, a curing agent and silicone oil according to a mass ratio of 11:1.2:2.5, uniformly;

Step 3: preparation of a continuous phase: preparing a second aqueous solution containing 8 wt % PVA;

Step 4 and Step 5 are the same as those in Embodiment 1, with a difference in that setting flow rates of the core phase, the shell phase and the continuous phase to 170 μL/h, 170 μL/h and 6200 μL/h, respectively; and

Step 6, preparation of microcapsules: collecting the double-emulsion drops obtained in Step 5 into a flask for water bath incubation at 45° C. for 3.5 h under a condition of 170 r/min.

### Embodiment 3 Preparation of Microcapsules

Step 1, preparation of a core phase: preparing a first aqueous solution containing 12 wt % PVA, 4 wt % sucrose and 0.3 wt % pollution-free food colorant, and adjusting the first aqueous solution to have an osmotic pressure of 2200 mOSM/L by adding 1 mol/L sodium chloride solution; Step 2, preparation of a shell phase: mixing PDMS, a curing agent and silicone oil according to a mass ratio of 9:0.8:3, uniformly;

Step 3: preparation of a continuous phase: preparing a second aqueous solution containing 12 wt % PVA;

Step 4 and Step 5 are the same as those in Embodiment 1, with a difference in that setting flow rates of the core phase, the shell phase and the continuous phase to 180 μL/h, 180 μL/h and 5800 μL/h, respectively; and

Step 6, preparation of microcapsules: collecting the double-emulsion drops obtained in Step 5 into a flask for water bath incubation at 55° C. for 2.5 h under a condition of 130 r/min.

### Test Embodiment 1 Mechanical Performance of Shell of Microcapsules

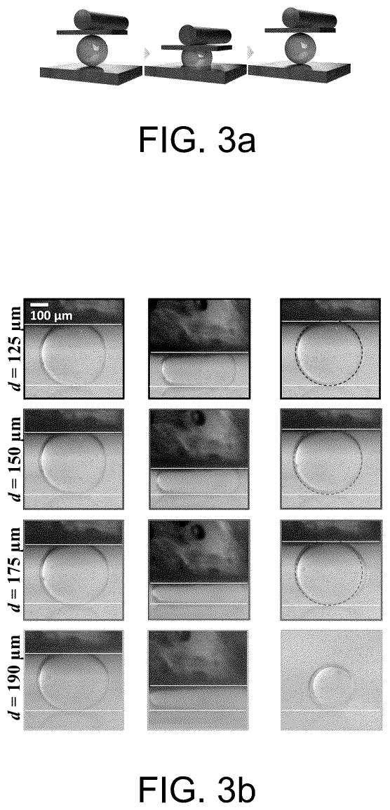

The inflated microcapsule allows its elastic shell to still maintain integrity and mechanical performance without any mechanical stress, and thus the active substance can be stored safely within the interior cavity without leakage. When deformation exceeds the critical value of mechanical rupture of the elastic shell, the microcapsules are ruptured, release the active substance of the core phase in milliseconds intensively, and stimulate the surrounding environment mechanically. In order to study its mechanical response in a quantitative way, a mechanical test was performed on a single microcapsule, shown as FIG. 3a. Mechanical force was applied to compress the inflated microcapsule and then removed, and whether the microcapsule can be restored to the original state was observed.

The microcapsules obtained in Step 6 of Embodiment 1 were transferred to an aqueous solution containing 2 wt % PVA, and the aqueous solution was adjusted to have an osmotic pressure difference ΔC of 1740 mOsml⁻¹ by sodium chloride solution, so that the microcapsules underwent a permeable inflation. The obtained microcapsules have an diameter of 271 μm, a shell thickness of 4 μm and an inflated degree α of 2.53. Then, the microcapsules were entirely soaked in water, and deformation was observed via camera while forces required for compression and relaxation were detected. A compression-relaxation rate was set to 1.4 μm s⁻¹ (equivalent to the strain rate of 0.005 s⁻¹), so as to eliminate a dynamic effect of compression.

Compression and relaxation of a single microcapsule were repeated four times, and meanwhile the greatest displacement d was increased from 125 μm to 150 μm, 175 μm and 190 μm. The SEM image of deformation process of the microcapsule is shown in FIG. 3b, and force-displacement curves of the microcapsule being compressed and relaxed are shown in FIG. 3c wherein the x-axis is a lateral displacement variation of the microcapsule. It can be seen that: (1) when the microcapsule underwent the greatest displacement d of 125 μm or a strain ε of 0.447, inapparent delaying was shown, that is, the microcapsule restored completely to a spherical shape after a cycle of compression and relaxation, and the force-displacement curve of being compressed overlaps the force-displacement curve of being relaxed; (2) when the microcapsule underwent a displacement d of 150 μm (a strain ε of 0.536) or a displacement d of 175 μm (a strain ε of 0.625), the microcapsule failed to restore to the original spherical shape after a cycle of compression and relaxation (the dotted line in FIG. 3c is a profile of the original spherical shape, indicating that the microcapsule after relaxation deviated from the original profile), and the force-displacement curve of being compressed and the force-displacement curve of being relaxed are in hysteresis wherein the slopes are increased with the increase of vertical displacement, since in order to avoid leakage, the microcapsule maintained an original volume of the interior cavity in the case of relatively great vertical displacement, thereby resulting greater lateral stretch; (3) when the microcapsule underwent a displacement d of 190 μm (a strain ε of 0.661), the microcapsule instantly retracted to an uninflated state after a cycle of compression and relaxation, and cracks on the shell of the microcapsule can be observed. Before rupture the compression force Fₚₒₚ is 89.2 mN, which is divided by a contact area between the microcapsule and a parallel panel to convert into a stress of 0.491 MPa. It is indicated that the inflated microcapsule has elastic deformation when dis 125 μm or less; when d ranges from 150 μm to 175 μm, the microcapsule has plastic deformation since a lateral strain of the shell exceeds a yield point, but allows the shell to still maintain integrity and avoids the leakage of pollution-free colorant; and when d is 190 μm or more, the shell of the microcapsule fails to maintain the integrity since the lateral strain of the shell exceeds a rupture point, and thus the microcapsule is ruptured and releases the pollution-free colorant of the core phase.

Tests of mechanical performance were performed on the microcapsules obtained in Step 6 of Embodiments 2 and 3 by using the same method, and the results are found to be similar to that of Embodiment 1, indicating that the microcapsule of the present invention can maintain the shell integrity and avoid the leakage of active substance from the core even under the pressure of d<190 μm.

### Test Embodiment 2 Ultrafast Release of Active Substance in the Microcapsule

The microcapsules obtained in Step 6 of Embodiment 1 were transferred to an aqueous solution containing 2 wt % PVA, and the aqueous solution was adjusted to have an osmotic pressure of 500 mOSM/L by adding a 120 mmol/L sodium chloride solution, so that the microcapsules underwent a permeable inflation in the environment having an osmotic pressure difference ΔC of 1740 mOsml⁻¹. The obtained microcapsules have a diameter of 271 μm, a shell thickness of 4 μm and an inflated degree α of 2.53.

12 individual microcapsules were compressed at an equivalent strain rate of 0.05 s⁻¹ till the microcapsules were ruptured, and threshold strain and stress were detected with a result shown in FIG. 3d (the horizontal line represent an average value). It can be seen that an average threshold strain εₚₒₚ causing rupture of the microcapsules is 0.75 with a CV value of 10% and an average threshold stress σₚₒₚ is 0.43 MPa with a CV value of 47%.

In order to measure the time scale of the rupture of microcapsules and study how the microcapsules restore to an uninflated state, the present test embodiment used a high-speed camera to observe the microcapsules, while using a pair of glass slides to compress the microcapsules. The results are shown in FIG. 3e (compression started as t=0 ms). It can be seen that: (1) the inflated microcapsules became disc-shaped under compression and ruptured at the thinnest point beyond the threshold strain; (2) within t=0 to 0.33 ms, the microcapsules significantly contracted and vigorously ejected water from the cavity; (3) within t=0.33 to 1 ms, the microcapsules further contracted, and at 1.67 ms, they almost returned to the original dimension of the uninflated microcapsules, and the ejection gradually stopped. This indicates that the microcapsule of the present invention can achieve ultrafast release of active substance within one millisecond.

Tests of ultrafast release of active substance in the microcapsule were performed on the microcapsules obtained in Step 6 of Embodiments 2 and 3 by using the same method, and the results are found to be similar to that of Embodiment 1, indicating that the microcapsule of the present invention can achieve ultrafast release of active substance within one millisecond.

### Test Embodiment 3 Effects of Inflated Degree of Elastic Shell of the Microcapsule on Mechanical Stimulation Resulted by Rupture

After the microcapsule is inflated, the shell of the microcapsule would be stretched so that the microcapsule is more sensitive to the external mechanical stress. After the microcapsule is ruptured, interior elastic potential energy thereof (including energy generated by permeable inflation and compression deformation) is converted into mechanical potential energy, to provide mechanical stimulation to the surrounding environment. In order to study effects of the inflated degree of microcapsule on its mechanical response in a quantitative way, the microcapsules obtained in Step 6 of Embodiment 1 were transferred to an aqueous solution containing 2 wt % PVA, then inflated degrees of 5 microcapsules were adjusted by adding sodium chloride solution, respectively, i.e. 1, 1.34, 1.63, 2.53 and 3.59, and the microcapsules were compressed at an equivalent strain rate of 0.05 s⁻¹.

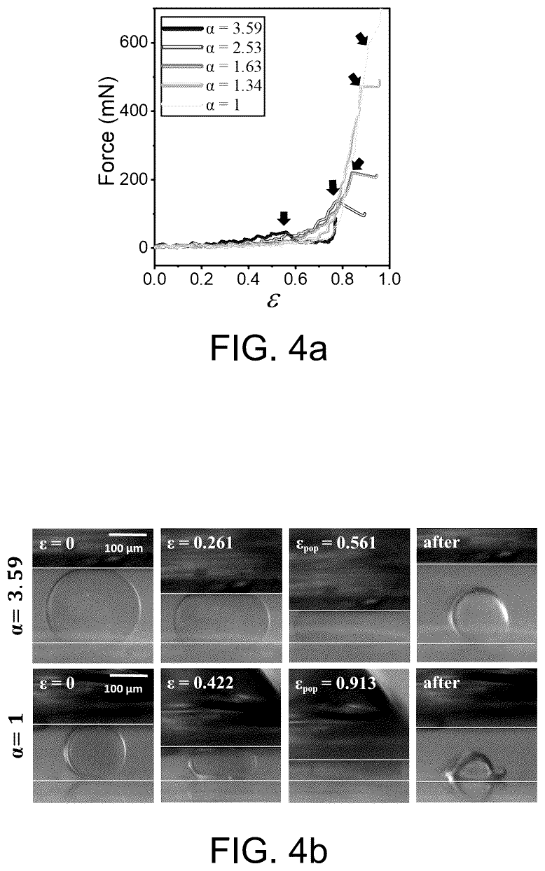

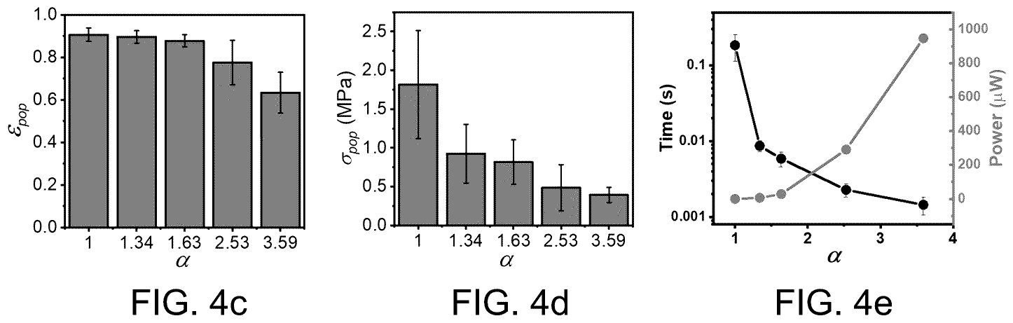

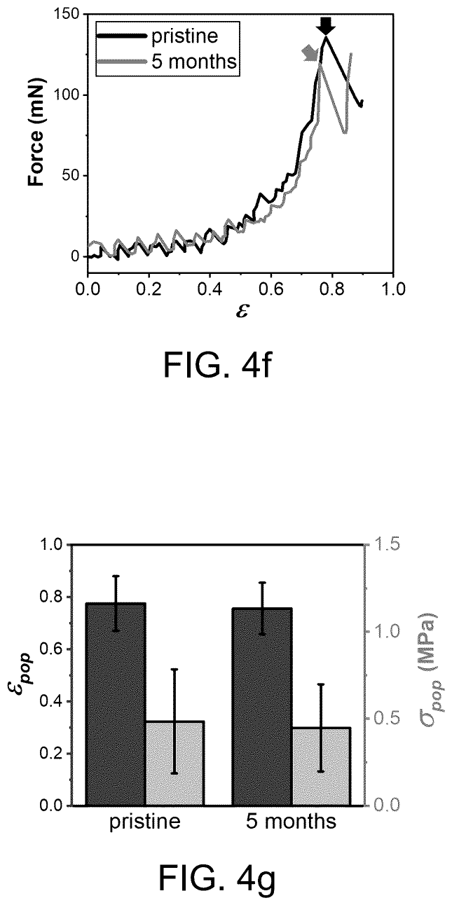

The obtained compression force-strain curve is shown in FIG. 4a (the moment of rupture is indicated by an arrow), the electron microscopy images of the deformation process of microcapsules with a values of 1 and 3.59 are shown in FIG. 4b (from uncompressed to compressed to rupture to removal of compression force), the average strain-inflated degree curve is shown in FIG. 4c, and the stress-inflated degree curve is shown in FIG. 4d. The effect of inflated degree on the rupture time (measured by monitoring the ejection behavior with a high-speed camera) and the mechanical potential energy obtained from the conversion of elastic potential energy stored by microcapsule inflation is shown in FIG. 4e.

It can be seen that, (1) for the microcapsule having α of 1.34 or less, the force didn't drop at the time of rupture; and for the microcapsule having α of 1.63 or more, the force suddenly dropped at the time of rupture due to the ejection of water from the cavity; (2) when when α=3.59, strain εₚₒₚ=0.561 and force Fₚₒₚ=47.2 mN at the time of rupture, the microcapsule rapidly retracted to the original dimension and shape in the uninflated state; (3) the average strain εₚₒₚ of microcapsule at the time of rupture was decreased from 0.900 to 0.877 when α increased from 1 to 1.63; when α reached to 3.59, the average strain εₚₒₚ of microcapsule at the time of rupture was dramatically decreased to 0.633; (4) the average stress (force divided by contact area) of microcapsule at the time of rupture was decreased from 1.82 MPa to 0.39 MPa when α increased from 1 to 3.59, indicating that the higher the inflated degree, the more sensitive the microcapsule to the exterior mechanical stress at the time of rupture, and that the inflated degree significantly affect the dynamics of inflation; (5) a rupture time reached to 184 ms when α=1, indicating that the release rate was not that fast without inflation of shell; the rupture time was 1.45 ms when α=3.59, and the rupture time was 8.68 ms when α=1.34; (6) it can be seen that the rupture time decreased with the increase of α; the mechanical power generated by the permeable inflation of microcapsules dramatically increased with the increase of α; the mechanical power generated by permeable inflation was 947 μW when α=3.59, which is 148 times when α=1.34; (7) the mechanical potential energy released by the rupture of microcapsules was much greater than that generated solely by permeable inflation, as the inflated microcapsules would further deform under compression, which also generated mechanical potential energy, allowing the microcapsules to store greater elastic potential energy during rupture.

It is indicated that when α is more than 1.63, especially ranging from 2.53 to 3.59, the release process of active substance in the microcapsule exerts a relatively strong mechanical stimulation intensity on the surrounding environment which can further improve an absorption efficiency to the active ingredients in the fields of pharmaceuticals and cosmetics, and can provide a unique sensual experience in the field of food.

Tests of effects of inflated degree of elastic shell of the microcapsule on mechanical stimulation resulted by rupture were performed on the microcapsules obtained in Step 6 of Embodiments 2 and 3 by using the same method, and the results are found to be similar to that of Embodiment 1, indicating that the microcapsule of the present invention can release the active substance quickly under the specific external force, and exerts a relatively strong mechanical stimulation intensity on the surrounding environment, and can further improve an absorption efficiency to the active ingredients in the fields of pharmaceuticals and cosmetics, and can provide a unique sensual experience in the field of food.

### Test Embodiment 4 Stability of Mechanical Stimulation Resulted by the Rupture of Microcapsule

Microcapsules having α of 2.53 were prepared by reference of the method in Test Embodiment 2 and stored at 25° C. for 5 months, and then compressed with the same strain as the microcapsules prepared on-site by the same method.

The obtained compression force-strain curves are shown in FIG. 4f (indicated by an arrow at the time of rupture), and measurement results of the average strain and stress are shown in FIG. 4g. It can be seen that the results of the microcapsules stored for 5 months are similar to those of the on-site prepared microcapsules, and there is no significant difference. It shows that when the external strain in the expanded microcapsule is lower than the yield point, the deformation is completely elastic, and long-term storage will not affect the mechanical properties of the shell.

That is, after long-term storage, it can still generate strong mechanical stimulation to the surrounding environment when ruptured, further improving the absorption efficiency of active ingredients and having excellent stability.

Tests of stability of mechanical stimulation resulted by the rupture of microcapsule were performed on the microcapsules obtained in Step 6 of Embodiments 2 and 3 by using the same method, and the results are found to be similar to that of Embodiment 1, indicating that the mechanical properties of the shell will not be affected during the long-term storage of the microcapsules. That is, after long-term storage, it can still generate strong mechanical stimulation to the surrounding environment when ruptured, further improving the absorption efficiency of active ingredients and having excellent stability.

### Test Embodiment 5 Sensual Usage Experience of the Microcapsule

The microcapsules obtained in Step 6 of Embodiment 1 were transferred to an aqueous solution containing 2 wt % PVA, and the microcapsules underwent a permeable inflation by adding sodium chloride solution. The obtained microcapsules have a diameter of 795 μm and an inflated degree α of 3.59.

The microcapsules were applied to the skin and gently rubbed back and forth with fingers for 15 cycles (with a shear rate of approximately 2.5 cm s⁻¹). It was found that the microcapsules are very soft and elastic, providing a comfortable touch to the skin. Rubbing can cause the microcapsules to be ruptured, not only releasing the pollution-free colorant (active substances) very quickly, but also making the skin perceive the mechanical energy generated by the rupture. In addition, there is no residual foreign body sensation on the skin after rupture.

Tests of sensual usage experience of the microcapsule were performed on the microcapsules obtained in Step 6 of Embodiments 2 and 3 by using the same method, and the results are found to be similar to that of Embodiment 1, indicating that the microcapsule of the present invention not only releases the pollution-free colorant (active substances) very quickly, but also makes the skin perceive the mechanical energy generated by the rupture. In addition, there is no residual foreign body sensation on the skin after rupture.

In conclusion, the microcapsules of the present invention are capable of not only keeping a relatively high mechanical stability without any mechanical stress and storing the active substance within the interior cavity without leakage, but also completely releasing the active substance in milliseconds when the mechanical stress exceeds the critical stress of rupture of the microcapsule, and meanwhile the release process exerts a relatively strong mechanical stimulation intensity on the surrounding environment which can further improve an absorption efficiency to the active substance in the fields of pharmaceuticals and cosmetics, and can provide a unique sensual experience in the field of food. Therefore, the microcapsules can serve as an excellent delivery carrier for active substance, and suitable for preparing cosmetics, pharmaceuticals and food.

The above embodiments are preferred implementation of the present invention, but the present invention is not limited by the above embodiments. Any other changes, modifications, substitutions, combinations, or simplifications that do not deviate from the spirit and principles of the present invention should be equivalent substitution methods and are included in the scope of protection of the present invention.

What is claimed is:

1. A microcapsule, comprising a core phase and a shell phase with a volume ratio of (3.4-3.8):(0.8-1.2);
wherein the core phase comprises 8 wt % to 12 wt % of PVA, 4 wt % to 6 wt % of a density enhancer, 0.1 wt % to 0.3 wt % of an active substance, and a residual amount of water; the shell phase comprises an elastomer precursor, a curing agent and silicone oil with a mass ratio of (9-11):(0.8-1.2):(2.5-3).

2. The microcapsule according to claim 1, wherein the density enhancer comprises at least one of sucrose, glucose, maltose, fructose, glycerol, and PEG.

3. The microcapsule according to claim 1, wherein the elastomer precursor is Polydimethylsiloxane (PDMS).

4. The microcapsule according to claim 1, wherein the curing agent is a reagent capable of curing the elastomer precursor.

5. The microcapsule according to claim 1, wherein the microcapsule has an inflated degree α more than 1.63.

6. A preparation method for the microcapsule according to claim 1, comprising:
preparing double-emulsion drops via a microfluidic device first, and then performing a heat treatment to obtain the microcapsule.

7. The preparation method according to claim 6, wherein the microfluidic device comprises an injection capillary, a collection capillary, and a square capillary at the outermost, the injection capillary and the collection capillary are inserted into both sides of the square capillary, respectively, and spaced apart to form an emulsifying region in which ends of the injection capillary and the collection capillary are cone-shaped.

8. The preparation method according to claim 7, wherein the core phase is injected through the injection capillary, the shell phase is injected through a gap between the injection capillary and the square capillary, and a continuous phase is injected through the gap between the collection capillary and the square capillary.

9. The preparation method according to claim 6, wherein the heat treatment is performed at 45° C. to 55° C. for 2.5 to 3.5 h.

10. Use of the microcapsule according to claim 1 in the fields of cosmetics, pharmaceuticals and/or food.

11. Use of the microcapsule according to claim 2 in the fields of cosmetics, pharmaceuticals and/or food.

12. Use of the microcapsule according to claim 3 in the fields of cosmetics, pharmaceuticals and/or food.

13. Use of the microcapsule according to claim 4 in the fields of cosmetics, pharmaceuticals and/or food.

14. Use of the microcapsule according to claim 5 in the fields of cosmetics, pharmaceuticals and/or food.

15. Use of the microcapsule according to claim 6 in the fields of cosmetics, pharmaceuticals and/or food.

16. Use of the microcapsule according to claim 7 in the fields of cosmetics, pharmaceuticals and/or food.

17. Use of the microcapsule according to claim 8 in the fields of cosmetics, pharmaceuticals and/or food.

18. Use of the microcapsule according to claim 9 in the fields of cosmetics, pharmaceuticals and/or food.
